(19) United States
(12) Patent Application Publication
(10) Pub. No.: US 2010/0055680 A1
(43) Pub. Date: Mar. 4, 2010

(54) GENOMIC DNA LABELING AND AMPLIFICATION

(75) Inventor: Shirley Jufang Shi, Lexington, MA (US)

Correspondence Address: GIFFORD, KRASS, SPRINKLE, ANDERSON;& CITKOWSKI, P.C., P.O. BOX 7021, TROY, MI, 48007-7021, US

(73) Assignee: PerkinElmer LAS, Inc., Waltham, MA (US)

(21) Appl. No.: 12/199,993

(22) Filed: Aug. 28, 2008

(51) Int. Cl. C12Q 1/68; C12P 19/34

(52) U.S. Cl. 435/6; 435/91.2

(57) ABSTRACT

Methods of amplifying genomic DNA are provided which include contacting template genomic DNA, a plurality of random primers, and a DNA polymerase, wherein the ratio of template genomic DNA to random primers is in the range of about 1:10-1:35,000,000 (w/w), inclusive, to produce a reaction mixture; and incubating the reaction mixture under isothermal conditions suitable for DNA synthesis, thereby producing amplified genomic DNA characterized by less than 10 percent gene copy number error. The template genomic DNA used in described methods can be in denatured condition and/or in non-denatured condition to achieve production of amplified genomic DNA characterized by less than 10 percent gene copy number error. In a particular option, the reaction mixture includes detectably labeled nucleotides and detectably labeled amplified genomic DNA characterized by less than 10 percent gene copy number error is produced.

### FIELD OF THE INVENTION

Methods and compositions described relate generally to generating copies of template DNA. Embodiments of methods and compositions described relate specifically to generating copies of template genomic DNA, wherein the copies are characterized by less than 10 percent gene copy number error and accurately reflect the gene copy number profile of the template genomic DNA, as well as to generating labeled copies of template genomic DNA.

### BACKGROUND OF THE INVENTION

Molecular genetic analysis is increasingly important as a tool in both clinical and research applications of biomedical sciences. The amount of genetic material available for such analytic procedures is often limited, for example when working with samples from prenatal, neonatal and infant subjects or with biopsy material such as needle aspirates from tumors and laser capture micro-dissected samples from heterogeneous tissues. In order to obtain sufficient amounts of genomic DNA, amplification techniques are used to make copies of a genome. Unfortunately, most previously reported methods of genome amplification often introduce significant sequence bias and numerous errors, making quantitative assays, such as genomic copy number analysis, difficult. Thus, there is a continuing need for methods of genome amplification without significant gene copy number error.

### SUMMARY OF THE INVENTION

Methods of amplifying genomic DNA are provided which include contacting template genomic DNA, a plurality of random primers, and a DNA polymerase, wherein the ratio of template genomic DNA to random primers is in the range of about 1:10-1:35,000,000 (w/w), inclusive, to produce a reaction mixture; and incubating the reaction mixture under isothermal conditions suitable for DNA synthesis, thereby producing amplified genomic DNA characterized by less than 10 percent gene copy number error.

The template genomic DNA used in described methods can be in denatured condition and/or in non-denatured condition to achieve production of amplified genomic DNA characterized by less than 10 percent gene copy number error.

In a particular option, the reaction mixture includes detectably labeled nucleotides and detectably labeled amplified genomic DNA characterized by less than 10 percent gene copy number error is produced.

In a further option, the reaction mixture includes detectably labeled random primers and detectably labeled amplified genomic DNA characterized by less than 10 percent gene copy number error is produced.

The template genomic DNA included in the reaction mixture can be derived from any source, including, but not limited to, a human, a non-human mammal, a vertebrate, an invertebrate, a microorganism, a plant, cultured cells and laboratory manipulated cells.

A template genomic DNA is amplified at least 3-fold using a method of amplification or a method of amplification and labeling described herein. In particular methods, the template genomic DNA is amplified at least 2,500-fold.

Methods of assaying genomic DNA are provided which include contacting template genomic DNA, random primers, and a DNA polymerase, wherein the ratio of template genomic DNA to random primers is in the range of about 1:10-1:35,000,000 (w/w), inclusive, to produce a reaction mixture; incubating the reaction mixture under isothermal conditions suitable for DNA synthesis, thereby producing amplified genomic DNA characterized by less than 10 percent gene copy number error; and comparing at least a portion of the amplified genomic DNA to a standard, thereby assaying genomic DNA. In a particular assay described herein, the amplified genomic DNA is compared to reference DNA to determine a number of copies of one or more regions in the amplified genomic DNA.

Methods of producing detectably labeled amplified genomic DNA are provided which include contacting genomic DNA template, random primers, dNTPs wherein at least one type of dNTP is detectably labeled, and a DNA polymerase, wherein the ratio of genomic DNA to random primers is in the range of about 1:10-1:35,000,000 (w/w), inclusive, to produce a reaction mixture; and incubating the reaction mixture under substantially isothermal conditions suitable for DNA synthesis, thereby producing detectably labeled amplified genomic DNA characterized by less than 10 percent gene copy number error.

### BRIEF DESCRIPTION OF THE DRAWINGS

FIG. 1A is a graph showing results of a parallel genomic hybridization (PGH) assay using amplified genomic DNA prepared as described herein, to demonstrate the >99 percent confidence of detecting single-copy gain of a chromosome; and

FIG. 1B is a graph showing results of a parallel genomic hybridization (PGH) assay using amplified genomic DNA prepared as described herein, to demonstrate the >99 percent confidence of detecting single-copy loss of a chromosomal region (s).

### DETAILED DESCRIPTION OF THE PREFERRED EMBODIMENTS

Methods are described herein for amplifying genomic DNA to produce copies characterized by the gene copy number profile of the template genomic DNA. Methods are also described herein for amplifying genomic DNA to produce detectably labeled copies characterized by the gene copy number profile of the template genomic DNA.

Scientific and technical terms used herein are intended to have the meanings commonly understood by those of ordinary skill in the art. Such terms are found defined and used in context in various standard references illustratively including J. Sambrook and D. W. Russell, Molecular Cloning: A Laboratory Manual, Cold Spring Harbor Laboratory Press; 3rd Ed., 2001; F. M. Ausubel, Ed., Short Protocols in Molecular Biology, Current Protocols; 5th Ed., 2002; B. Alberts et al., Molecular Biology of the Cell, 4th Ed., Garland, 2002; D. L. Nelson and M. M. Cox, Lehninger Principles of Biochemistry, 4th Ed., W. H. Freeman & Company, 2004; Maliga, P., Methods in Plant Molecular Biology, Cold Spring Harbor Laboratory Press, New York, 1995; and Herdewijn, P. (Ed.), Oligonucleotide Synthesis: Methods and Applications, Methods in Molecular Biology, Humana Press, 2004.

A method of amplifying genomic DNA is described which includes contacting genomic DNA template, random sequence oligonucleotide primers, and a DNA polymerase to produce a reaction mixture. The reaction mixture is incubated under substantially isothermal conditions suitable for DNA synthesis. Surprisingly, it is found that amplified genomic DNA and/or labeled amplified genomic DNA is prepared according to methods described herein using scant biological samples, and the amplified genomic DNA and/or labeled amplified genomic DNA is characterized by less than 10 percent gene copy number error and accurately reflects the gene copy number profile of the template genomic DNA.

In a further unexpected aspect, it is found that heat denaturation of the genomic DNA template is not necessary to produce labeled or unlabeled amplified genomic DNA using methods described herein. The term “denaturation,” also known as “melting,” in reference to genomic DNA refers to disruption of hydrogen bonds between complementary nucleotide bases of complementary DNA strands. In particular methods described herein, the template genomic DNA is in a non-denatured state when added to the reaction mixture and is not subjected to heat denaturation in the reaction mixture or during incubation of the reaction mixture. Without wishing to be bound by theory, it is believed that primer template DNA ratios used in methods described herein contribute to disruption of hydrogen bonds between complementary nucleotide bases of the template genomic DNA in the reaction mixture.

In further particular methods described herein, the template genomic DNA is not subjected to chemical or enzymatic fragmention prior to inclusion in the reaction mixture, prior to incubation of the reaction mixture or during incubation of the reaction mixture.

The genomic DNA template can be obtained from any source, including, but not limited to, a human, a non-human mammal, a vertebrate, an invertebrate, a microorganism, or a plant. Genomic DNA template can be obtained from one or more cells ex vitro or in vitro. For example, genomic DNA template can be obtained from cultured cells, including, but not limited to, cell lines, primary cells or laboratory manipulated cells such as recombinant cells.

The genomic DNA template in the reaction mixture can be in any of various forms, including, but not limited to, double helical, supercoiled, relaxed, single stranded, double stranded, fragmented or a combination of forms. The genomic DNA template can have one or more blunt ends and/or one or more overhanging ends. Optionally, the genomic DNA template is modified such as by heat denaturation, chemical treatment and/or enzymatic fragmentations, although such treatment is not required.

In particular methods described herein, at least one substantially whole genome derived from any source, such as a human, a non-human mammal, a vertebrate, an invertebrate, a microorganism, a plant, cultured cells or laboratory manipulated cells is included in the reaction mixture as genomic DNA template.

For example, genomic DNA can be isolated from a single cell and the isolated genomic DNA, representative of a single genome, can be used as template genomic DNA in methods described herein. In a further example, genomic DNA can be isolated from multiple cells and an amount of the isolated genomic DNA, representative of a single genome or multiple genomes, can be used as template genomic DNA in methods described herein.

In one example, genomic DNA can be one or more individual chromosomes. An isolated chromosome, that is, DNA representative of a single chromosome, can be used as template genomic DNA in methods described herein. In a further example, genomic DNA can be isolated from more than one chromosome of the same type, such as multiple X chromosomes, multiple Y chromosomes or multiple somatic chromosomes such as multiples of chromosome 1, 2, 3, 4, 5, 6, 7, 8, 9, 10, 11, 12, 13, 14, 15, 16, 17, 18, 19, 20, 21 or 22. Isolated chromosomal DNA, that is, DNA representative of more than one chromosome of the same type or different types, can be used as template genomic DNA in methods described herein.

In a further example, genomic DNA can be one or more regions of individual chromosomes. One or more isolated chromosome regions can be used as template genomic DNA in methods described herein. In another example, genomic DNA can be one or more regions of individual chromosomes containing one or more genes. One or more isolated chromosome regions containing one or more genes can be used as template genomic DNA in methods described herein.

In one example, genomic DNA can be isolated from a single mitochondrion and the isolated genomic DNA, representative of a single mitochondrion, can be used as template genomic DNA in methods described herein. In a further example, genomic DNA can be isolated from multiple mitochondria and an amount of the isolated genomic DNA, representative of a single mitochondrion or multiple mitochondria, can be used as template genomic DNA in methods described herein.

Genomic DNA template for use in a method described herein is obtained by any of various techniques well-known in the art, exemplified by techniques described in J. Sambrook and D. W. Russell, Molecular Cloning: A Laboratory Manual, Cold Spring Harbor Laboratory Press; 3rd Ed., 2001; F. M. Ausubel, Ed., Short Protocols in Molecular Biology, Current Protocols; 5th Ed., 2002; B. Alberts et al., Molecular Biology of the Cell, 4th Ed., Garland, 2002; D. L. Nelson and M. M. Cox, Lehninger Principles of Biochemistry, 4th Ed., W. H. Freeman & Company, 2004; and Maliga, P., Methods in Plant Molecular Biology, Cold Spring Harbor Laboratory Press, New York, 1995.

The term “primer” refers to a single stranded oligonucleotide which serves as a point of initiation for template-directed DNA synthesis under conditions suitable for DNA synthesis.

One of skill in the art will recognize conditions suitable for template-directed DNA synthesis depend on factors such as length of the primer, buffer, pH, Mg salt concentration and temperature. In general, components of the reaction mixture are in aqueous solution.

Methods described herein for amplifying genomic DNA to produce copies characterized by the gene copy number profile of the template genomic DNA without significant copy number error include reaction mixture conditions having incubation times of about 15 min-3 hours, dNTP concentration of about 0.6-2 mM for each dNTP, Mg concentration of about 2.5-7.5 mM, pH in the range of about 6.8-8.2. Longer incubation (>>3 hours), higher dNTP concentration (>>2 mM), pH significantly lower or higher than 7.5 (>pH8.2, or <<pH6.8), and higher Mg concentrations (>>5 mM) will give similar or even higher fold amplification, however, the fidelity of genomic DNA template gene copy number profile is not achieved and such conditions produce significant gene copy error.

Particular methods described herein include incubation of a reaction mixture for template-directed DNA synthesis under substantially isothermal conditions suitable for template-directed DNA synthesis. The temperature selected is a temperature suitable for template-directed DNA synthesis and is a temperature compatible with both the DNA polymerase used, as well as annealing temperature for primer-DNA template duplexes in the reaction mixture.

The DNA polymerase used can be a thermophilic DNA polymerase or a non-thermophilic DNA polymerase. In particular non-limiting examples, a non-thermophilic DNA polymerase included in the reaction mixture is a T7 DNA polymerase, SEQUENASE, an E. coli DNA polymerase I, an E. coli DNA polymerase I large fragment (Klenow), an exo-Klenow, a phi29 DNA polymerase or a T4 DNA polymerase. In particular non-limiting examples, a thermophilic DNA polymerase included in the reaction mixture is a Taq DNA polymerase, a Pfu DNA polymerase, DNA polymerases from Thermococcus litoralis such as described in U.S. Pat. Nos. 5,210,036; 5,500,363; and 5,352,778, VENT DNA polymerase, VENT-EXO DNA polymerase, DEEP VENT DNA polymerase or DEEP VENT EXO DNA polymerase. Combinations of DNA polymerases can be used. Temperatures suitable for template-directed DNA synthesis and compatible with a selected DNA polymerase are well-known in the art.

An annealing temperature for primer-DNA template duplexes in a reaction mixture is determined according to methods well-known in the art.

The term “isothermal conditions” refers to substantially constant temperature at which the reaction mixture is incubated to carry out template-directed DNA synthesis. One of skill in the art will appreciate that minor fluctuations in temperature may occur during incubation of a reaction mixture to carry out template-directed DNA synthesis. Thus, incubation under isothermal conditions is intended to indicate that the reaction mixture is not subjected to large changes in incubation temperature such as deliberately occur in thermal cycling for polymerase chain reactions.

Nucleotides, including, but not limited to, deoxynucleotide triphosphates (dNTPs) and analogs thereof, detectably labeled or unlabeled, are included in a reaction mixture for template-directed DNA synthesis. The term “nucleotide analog” in this context refers to a modified or non-naturally occurring nucleotide, particularly nucleotide analogs which can be polymerized, with or without naturally occurring nucleotides, by template directed DNA synthesis catalyzed by a DNA polymerase. Nucleotide analogs are well-known in the art. Particular nucleotide analogs are capable of Watson-Crick pairing via hydrogen bonds with a complementary nucleotide and illustratively include, but are not limited to, those containing an analog of a nucleotide base such as substituted purines or pyrimidines, deazapurines, methylpurines, methylpyrimidines, aminopurines, aminopyrimidines, thiopurines, thiopyrimidines, indoles, pyrroles, 7-deazaguanine, 7-deazaadenine, 7-methylguanine, hypoxanthine, pseudocytosine, pseudoisocytosine, isocytosine, isoguanine, 2-thiopyrimidines, 4-thiothymine, 6-thioguanine, nitropyrrole, nitroindole, and 4-methylindole. Nucleotide analogs include those containing an analog of a deoxyribose such as a substituted deoxyribose, a substituted or non-substituted arabinose, a substituted or non-substituted xylose, and a substituted or non-substituted pyranose. Nucleotide analogs include those containing an analog of a phosphate ester such as phosphorothioates, phosphorodithioates, phosphoroamidates, phosphoroselenoates, phosophoroanilothioates, phosphoroanilidates, phosphoroamidates, boronophosphates, phosphotriesters, and alkylphosphonates such as methylphosphonates.

Random sequence primers, also called random primers or universal random primers, are included in a reaction mixture for amplification of genomic DNA template using methods described herein. The terms “random sequence primers”, “universal random primers”, “random primers” are used interchangeably to refer to oligonucleotide primers having sequences that are not necessarily specifically designed to be identical or complementary to a specified region of the genomic DNA template. The terms “random sequence primers”, “universal random sequence primers” and “random primers” are used to describe a population of primers, many or all of which have different sequences, of which some, or all, of the random primers will hybridize to a genomic DNA template based on statistical likelihood under a given set of hybridization conditions. Random primers can be obtained commercially or can be obtained by methods such as chemical synthesis, recombinant generation or fragmentation of a larger nucleic acid.

The nucleotide compositions of the random primers can include naturally occurring nucleotides and/or nucleotide analogs, with or without detectable labels. Optionally, random primers include or are entirely composed of “universal nucleotide analogs” including universal bases with non-traditional base pairing properties. The term “universal nucleotide analog” refers to a nucleotide analog including a nucleotide base which forms a “base pair” with at least two, at least three or all four of the naturally occurring nucleotide bases. Universal nucleotide analogs are well-known in the art.

Random primers typically have a length in the range of about 5-100, inclusive, nucleotides, although random primers used in methods described herein can be smaller or larger. In particular methods described herein, random primers have a length in the range of about 5-25, inclusive, nucleotides. In further methods described herein, random primers have a length in the range of about 8-14, inclusive, nucleotides.

The ratio of genomic DNA template to random primers in the reaction mixture is in the range of about 1:10-1:35,000,000 (w/w), inclusive, such as in the range of about 1:100-1:3,500,000; in the range of about 1:1000-135,000; in the range of about 1:50-1:20,000, each range being inclusive. In particular methods described herein, the ratio of genomic DNA template to random primers in the reaction mixture is in the range of about 1:50-1:20,000 (w/w), inclusive. Increased amplification fold is achieved with low input genomic DNA, that is, a ratio of genomic DNA template to random primers in the range of about 1:20,000-1:35,000,000 (w/w), inclusive.

The amount of genomic DNA template is present in the reaction mixture is not limited to particular amounts. In certain applications, genomic DNA template is present in an amount representative of at least one genome of a cell or organism. Particular methods described herein include genomic DNA template in the range of about 3 pg-1.5 μg, inclusive, such as about 3 pg-30 ng, inclusive; 30 ng-300 ng, inclusive, and 300 ng-1.5 μg, inclusive.

Optionally, amplified genomic DNA is used as genomic DNA template. For example, genomic DNA isolated from a single cell, a small number of cells or limited cell clusters can be amplified as described herein and the amplified DNA can be used as a template in a subsequent amplification reaction.

Amplified DNA produced according to a method described herein is useful in various genomic analyses such as fluorescent in situ hybridization (FISH), sequencing, single nucleotide polymorphism (SNP), gene expression analysis, gene expression genome association analysis, comparative genomic hybridization (CGH), and parallel genomic hybridization (PGH), as sometime required by patients or clinicians for decision making.

Amplified DNA produced according to a method described herein can be used in prenatal genetic/genomic diagnostics; preimplantation diagnostics, for instance using template material derived from single cells such as a single polar body cell; and disease-related assays, such as cancer genetic/genomic diagnostics.

Optionally, a reaction mixture includes detectably labeled nucleotides, detectably labeled nucleotide analogs and/or detectably labeled random primers to generate delectably labeled amplified genomic DNA. Nucleotides, such as dNTPs, nucleotide analogs and/or primers can be labeled directly or indirectly with a detectable label.

The term “detectable label” refers to a substance that can be measured and/or observed, visually or by any appropriate method illustratively including spectroscopic, optical, photochemical, biochemical, enzymatic, electrical and/or immunochemical methods of detection, to indicate presence of the label. Non-limiting examples of detectable labels that can be used in conjunction with methods described herein illustratively include a fluorescent moiety, a chemiluminescent moiety, a bioluminescent moiety, a magnetic particle, an enzyme, a substrate, a radioisotope and a chromophore. For example, dNTPs, nucleotide analogs and/or primers can be labeled with a dye, such as a fluorophore, a chromophore, a radioactive moiety or a member of a specific binding pair such as biotin. The term “member of a specific binding pair” refers to a substance that specifically recognizes and interacts with a second substance exemplified by specific binding pairs such as biotin-avidin, biotin-streptavidin, antibody-antigen, and target-aptamer. Non-limiting examples of detectable labels that can be used include fluorescent dyes such as fluorescein, fluorescein isothiocyanate, rhodamine, rhodamine isothiocyanate, Texas Red, cyanine dyes such as Cyanine 3 and Cyanine 5, and ALEXA dyes; chromophores such as horseradish peroxidase, alkaline phosphatase and digoxigenin; and radioactive moieties such as ³²P, ³⁵S, ³H, ¹²⁵I, or ¹⁴C; and binding partners such as biotin and biotin derivatives. Methods for detectably labeling dNTPs, nucleotide analogs and/or primers are well-known in the art.

The term “amplified genomic DNA” refers to the product of a process of copying a genomic DNA template. Methods described herein produce amplified genomic DNA characterized by less than 10 percent gene copy number error, including less than 5 percent gene copy number error. Methods described herein allow a user to confidently detect single-copy gain or loss of a chromosome, or a region of chromosome, and/or specific gene or genes. The term “gene copy number error” refers to a consequence of biased copying of genomic template DNA resulting in over-representation or under-representation of particular gene sequences in amplified genomic DNA. Thus, methods described herein produce amplified genomic DNA characterized by high fidelity of gene copy number profile of an original genomic DNA template and allow for quantitative determination of true gene copy number profile in a genomic DNA template.

Amplified genomic DNA characterized by high fidelity of gene copy number and less than 10 percent gene copy number error is not necessarily identical to the genomic DNA template. For example, amplified genomic DNA is detectably labeled and/or includes nucleotide analogs such that the amplified genomic DNA is non-identical to the genomic DNA template while still characterized by high fidelity of gene copy number and less than 10 percent gene copy number error.

Methods described herein achieve at least 3-fold amplification of the genomic DNA template. Further methods described herein achieve at least 2500-fold amplification of the genomic DNA template, including at least 10-fold, at least 50-fold, at least 100 fold, at least 1000 fold amplification.

Methods of assaying genomic DNA of a subject are described herein that include contacting genomic DNA template, random sequence oligonucleotide primers, and a DNA polymerase to produce a reaction mixture. The term “contacting” broadly refers to placing reaction mixture components together in a reaction vessel. Two or more of the reaction mixture components can be mixed prior to contacting all the components in the reaction vessel. The reaction mixture is incubated under substantially isothermal conditions suitable for DNA synthesis to produce amplified genomic DNA.

In a particular example, the amplified genomic DNA is compared to a reference DNA by hybridization assay such as comparative genomic hybridization (CGH) or parallel genomic hybridization (PGH). Broadly described, comparative genomic hybridization (CGH) refers to a hybridization reaction in which a test DNA sample from a subject and a reference DNA sample from a normal control are each differentially labeled with a detectable label, mixed, and then hybridized to probes such as normal metaphase chromosomes or, for array- or matrix-CGH, to a slide containing defined DNA probes. The ratio of signals from the two detectable labels is determined and analyzed to identify differences between the test DNA sample and the reference DNA sample.

In a particular example of CGH, a test DNA sample from a subject and a reference DNA sample are each labeled with a different color fluorescent tag. After mixing subject and reference DNA along with unlabeled human cot 1 DNA to suppress repetitive DNA sequences, the mix is hybridized to normal metaphase chromosomes or, for array- or matrix-CGH, to a slide containing hundreds or thousands of defined DNA probes. For example, the ratio of the two fluorescence signals along the chromosomes is used to evaluate regions of DNA gain or loss in the test sample.

Broadly described, PGH refers to a method in which labeled test sample DNA is hybridized with nucleic acid probes in parallel, but not in the same solution in which labeled reference sample DNA is hybridized with the nucleic acid probes. Again, the signals from the two detectable labels are compared and analyzed to identify differences between the test DNA sample and the reference DNA sample.

For example, in a PGH assay, hybridization of a test sample of fluorescently labeled template genomic DNA with DNA probes is performed in parallel with hybridization of fluorescently labeled reference DNA sample and DNA probes. Although there is no co-hybridization between the two fluorescently labeled DNA samples, the ratio of the two fluorescence signals (which can be the same color using PGH) is used to evaluate regions of DNA gain or loss in the test sample compared to the reference sample.

Methods of producing detectably labeled amplified genomic DNA are described herein which include contacting genomic DNA template, dNTPs, random primers, and a DNA polymerase in a reaction mixture. In particular methods, at least one type of dNTP is detectably labeled so that the produced amplified genomic DNA is detectably labeled by incorporation of the detectably labeled dNTP. The term “type of dNTP” refers to one of dATP, dCTP, dTTP, dGTP and analogs thereof.

In particular methods for amplifying genomic DNA template, the amplification reaction can be accomplished in a single reaction step. Thus, for example, the template and reaction components are added in a reaction vessel and incubated in the reaction vessel to produce the amplified genomic DNA. Similarly, in particular methods for amplifying and labeling genomic DNA template, the template is amplified and labeled in a single reaction step to produce labeled amplified genomic DNA.

In particular methods for amplifying genomic DNA template, the amplification reaction is accomplished in a single reaction vessel. Thus, for example, the template and reaction components are added in a reaction vessel and incubated in the reaction vessel to produce the amplified genomic DNA. Similarly, in particular methods for amplifying and labeling genomic DNA template, the template is amplified and labeled in a single reaction vessel to produce labeled amplified genomic DNA. Any suitable reaction vessel can be used, illustratively including, but not limited to, a well of a multi-well plate and a reaction tube.

Embodiments of compositions and methods described herein are illustrated in the following examples. These examples are provided for illustrative purposes and are not considered limitations on the scope of inventive compositions and methods.

### EXAMPLES

### Example 1

Isolation of Genomic DNA Template

Genomic DNA can be isolated by any commercial DNA purification kit from various suppliers, preferably Qiagen kit. Optionally, cells or specimens for use as a source of genomic DNA template are suspended in a buffer containing ribonuclease A. The cells are lysed by standard alkaline lysis techniques followed by centrifugation of the lysate at about 20,000 g for 30 minutes. The supernatant, containing the genomic DNA, is collected. The genomic DNA is extracted and purified from the supernatant by ethanol precipitation and centrifugation. The genomic DNA is resuspended in water or Tris-EDTA buffer.

### Example 2

Amplification of Genomic DNA Template

Test genomic DNA samples and reference DNA samples can be at a concentration of about 0.1 ng/microliter-50 ng/microliter. Twenty-four microliters of test and reference DNA is aliquoted into separate reaction microfuge tubes along with 20 microliters of random sequence oligonucleotide primer mix containing 1400 micrograms/ml random octamers, 125 mM Tris, 12.5 mM MgCl₂, and 25 mM 2-mercaptoethanol. Five microliters of a 10× dNTP mix containing: 1.2 mM each DATP, dGTP, dCTP and dTTP, 10 mM Tris 8.0, 1 mM EDTA, is added to each tube. An aliquot of exo-Klenow DNA polymerase containing 10-50 units, is added to each tube. The total volume of each tube is brought to a total of fifty microliters with water if necessary. The tubes containing the reaction mixture are then sealed and incubated at 37° C. for 1-2 hours. Amplified genomic DNA is quantified by measuring absorbance at 260 nm to determine the “fold” amplification.

### Example 3

Simultaneous Labeling and Amplification of Genomic DNA Template

Test genomic DNA samples and reference DNA samples can be about 0.1 ng/microliter-50 ng/microliter. Twenty-four microliters of test and reference DNA is aliquoted into separate reaction microfuge tubes along with 20 microliters of random sequence oligonucleotide primer mix containing 1400 micrograms/ml random octamers, 125 mM Tris, 12.5 mM MgCl₂, and 25 mM 2-mercaptoethanol. Optionally, the mixture can then be heated to 95-98 degrees C. and then allowed to cool to 4 degrees C. Five microliters of a 10× DNTP mix containing: 1.2 mM each of dATP, dGTP, and dTTP, 0.6 mM dCTP; 0.6 mM biotin-dCTP; 10 mM Tris 8.0; and 1 mM EDTA, is added to each tube. An aliquot of exo-Klenow DNA polymerase containing 10-50 units, is added to each tube. The total volume of each tube is brought to a total of fifty microliters with water if necessary. The tubes containing the reaction mixture are then sealed and incubated at 37° C. for 1-2 hours. Amplified labeled genomic DNA is quantified by measuring absorbance at 260 nm to determine the “fold” amplification.

### Example 4

Magnitude of Amplification of Genomic DNA Template

Test genomic DNA samples and reference DNA samples having about 3 picograms-1.5 micrograms of DNA can be at a concentration of about 0.001 ng/microliter-100 ng/microliter A desired amount of DNA in about twenty-four microliters of test and reference DNA is aliquoted into separate reaction microfuge tubes along with 20 microliters of random sequence oligonucleotide primer mix containing 1400 micrograms/ml random octamers, 125 mM Tris, 12.5 mM MgCl₂, and 25 mM 2-mercaptoethanol. Optionally, the mixture can then be heated to 95-98 degrees C. and then allowed to cool to 4 degrees C. Five microliters of a 10× dNTP mix containing: 1.2 mM each cATP, dGTP, dCTP and dTTP, 10 mM Tris 8.0, 1 mM EDTA, is added to each tube. An aliquot of exo-Klenow DNA polymerase containing 10-50 units, is added to each tube. The total volume of each tube is brought to a total of fifty microliters with water if necessary. The tubes containing the reaction mixture are then sealed and incubated at 37° C. for 1-2 hours. Amplified genomic DNA is quantified by measuring absorbance at 260 nm to determine the “fold” amplification. Measurements show amplification using one microgram of genomic DNA template produces about 3-5 fold amplification of the template. Amplification using 1 nanogram of genomic DNA template produces about 2000 fold amplification. A reaction using 15 picogram of genomic DNA produces about 100,000 fold amplification. When input DNA is very low such as 15 picogram genomic DNA, 5 ul or 12.5 ul total reaction volume is recommended, in order to achieve consistent amplification from run to run.

### Example 5

Assay Using Amplified Genomic DNA

Labeled amplified test and reference genomic DNA is purified away from remaining reaction mixture components, for instance by column purification or ethanol precipitation.

The purified labeled amplified test and reference genomic DNA is adjusted to a concentration of about 50-400 nanograms/microliter for each test or reference sample for parallel genomic hybridization. In this parallel genomic hybridization assay, characterized genomic sequence probes are affixed to a solid support in the form of beads. The biotin labeled amplified test is hybridized to the characterized genomic sequence probes on the beads, washed under high stringency conditions and then sequence specific binding of labeled amplified test to the characterized genomic sequences on the solid support is detected by incubating the hybrids with a detection reagent, PhycoLink SA, a commercially available phycoerythrin-conjugated streptavidin. In parallel, reference genomic DNA will undergo the same process of hybridization and reporter binding, but in a different well of same plate.

A hybridization buffer used contains Cot-1 DNA, formamide, dextran sulfate and 2× SSC. The labeled amplified test, in parallel with labeled reference genomic DNA, is hybridized to the characterized genomic sequences on the beads in a total volume of approximately 15 microliters in the wells of a rigid PCR-type microplate, such as the Bio-Rad HSP 9631 (Bio-Rad Laboratories, Hercules Calif.). The plate is sealed tightly to minimize evaporation using an aluminum foil sealer (MSF 1001, Bio-Rad). The hybridization incubation is performed overnight at 50° C. in a microplate shaking incubator at 1150 rpm (Wallac NCS Incubator, PerkinElmer).

After the hybridization, a hybridization wash is performed followed by incubation with a fluorescent reporter and a reporter wash. First, 100 microliters of wash buffer a (2×SSC, 50 percent formamide) is added to each well, the plate resealed and incubated in the shaking incubator with 1150 rpm agitation at 50° C. for 20 minutes. The content of each well is then transferred to a Millipore 0.46 μm HT filter plate (Millipore, Billerica Mass.). The liquid is then removed from each well by vacuum using a Millipore MSVMHTS00 vacuum manifold.

One hundred microliters of 1× PhycoLink SA solution, the streptavidin-phycoerythrin reporter is then added to each well. This reporter solution is made from 2 μl 500× PhycoLink SA PJ31S (Prozyme, San Leandro Calif.) mixed into 1 ml of reporter diluent, where the diluent is 1× PBS, 0.1% BSA and 0.05% Tween 20. This reporter solution is incubated with the hybrids for 30 minutes at 25° C. and 1150 RPM in the shaking incubator. After incubation, the solution is aspirated from the wells of the filter plate using the vacuum manifold as in the previous wash steps.

The hybrids are then washed once with wash buffer: 1× PBS with 0.01% Tween 20. One hundred microliters of wash buffer is added to each well of the filter plate, then the liquid is vacuum aspirated through the filters in the bottoms of the plate wells. One hundred microliters of the same wash buffer is added to re-suspend the beads for reading of a the detectable label by Luminex. For uniformity of suspension, incubation in the shaking incubator for 2 minutes at 25° C. at 1150 RPM is recommended.

A Luminex 200 system (Luminex Corporation, Austin Tex.) is used to detect the signal indicative of specific hybridization of the amplified genomic DNA or the standards. The median fluorescence intensity is recorded for each well of sample or reference and output in a data file. Normalized fluorescence signal ratio (FIR) between a sample to a standard is indicative of gene copy number profile of the sample, based upon the assumption of a standard as normal diploid across whole genome.

### Example 6

Determination of Gene Copy Number Profile Fidelity

A sample of genomic DNA template is amplified and simultaneously labeled as described above. Parallel genomic hybridization (PGH) is then performed using the resulting labeled amplified DNA and a second non-amplified labeled sample of the same genomic DNA template. Statistical analysis is performed and indicates that the amplified genomic DNA is characterized by high fidelity of gene copy number profile and less than 10% gene copy number error.

### Example 7

Samples of genomic DNA template from normal human males and normal human females are amplified and simultaneously labeled as described above. Parallel genomic hybridizations are then performed using the resulting labeled amplified DNA to analyze normal female/female, normal male/male, normal female/male and vice versa hybrids across 60 different genomic clones. Statistical analysis is performed and indicates that the amplified genomic DNA is characterized by high fidelity of gene copy number profile with less than 10% gene copy number error, and reliably detect gene copy number profile of X or Y-chromosome between male and female.

### Example 8

Samples of genomic DNA template from human samples having known trisomies are amplified and simultaneously labeled as described above. Parallel genomic hybridizations are then performed using the resulting labeled amplified DNA to analyze the test material compared to standard reference DNA. Statistical analysis is performed and indicates that the amplified genomic DNA is characterized by high fidelity of gene copy number profile with less than 10% gene copy number error and shows the expected chromosome gains, evidenced by elevated hybridization signal intensity ratios.

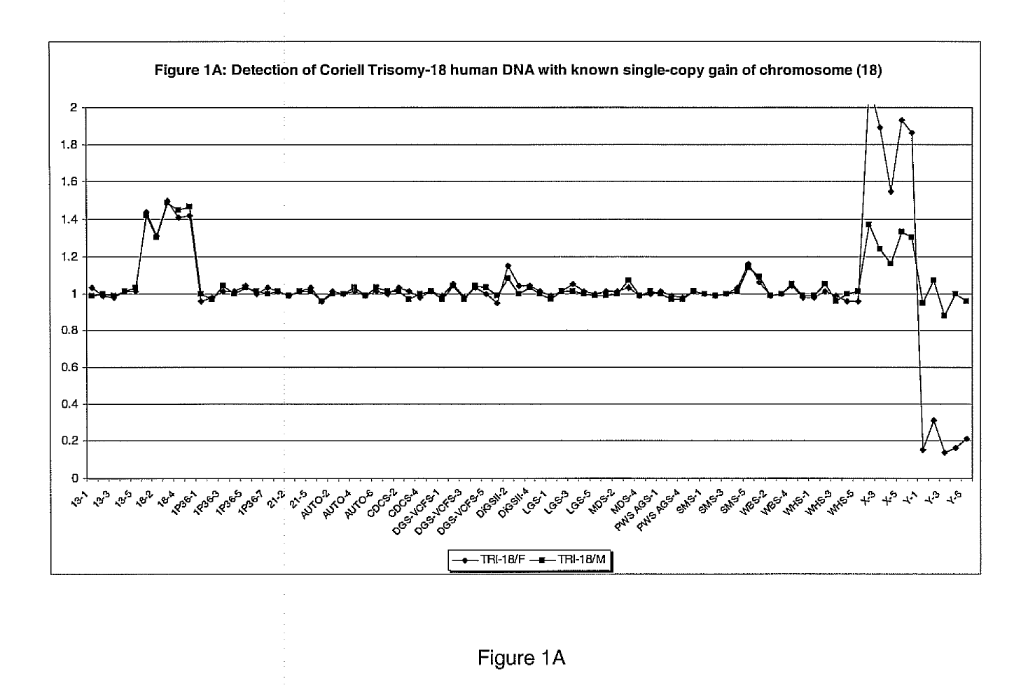

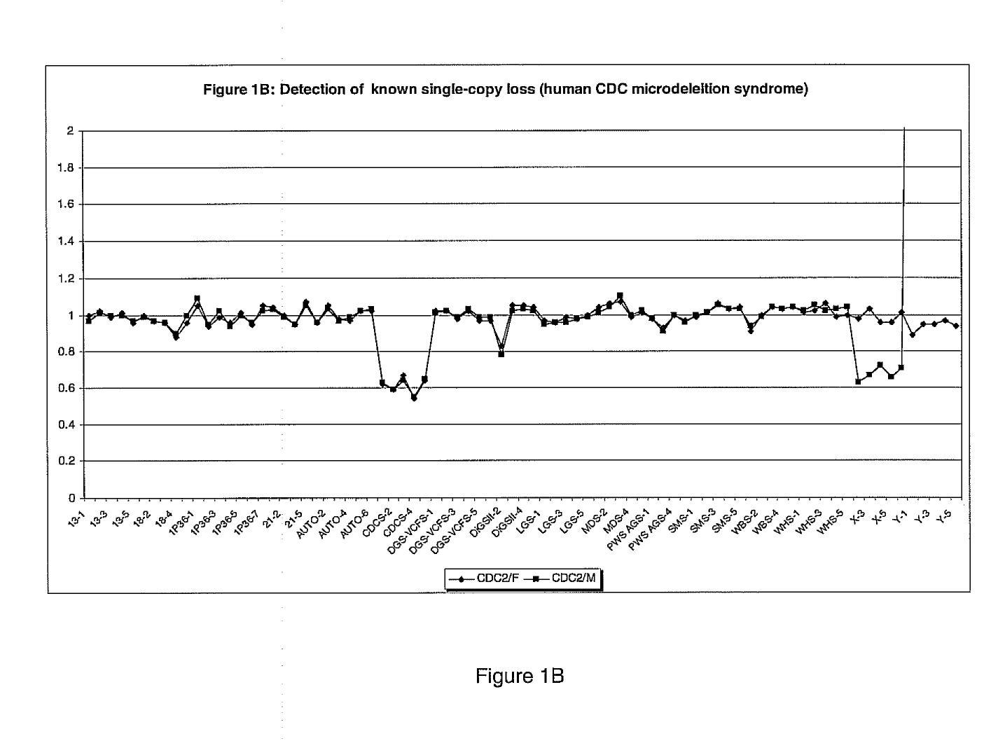

In a particular example, samples of genomic DNA template having known trisomies (trisomies 18 human DNA from Coriell Institute) were amplified and simultaneously labeled. Parallel genomic hybridizations were then performed using the resulting labeled amplified DNA. The ratios of hybridization signals of a sample against two references (normal male and normal female) were calculated and normalized. FIG. 1A is a graph showing results of a parallel genomic hybridization (PGH) assay using amplified genomic DNA prepared as described herein, to demonstrate the >99% confidence of detecting single-copy gain for the sample of Trisomy-18. The graph shows that elevated ratios across all BAC clones derived from chromosome 18 were observed (1.47 indicative of single copy of chromosome gain), while DNA BAC clones derived from all other regions have ratio of 1 (indicative of equal number of chromosome), except sex chromosome. FIG. 1A: X-axis: BAC DNA probes (chromosomal regions carried by bacterial artificial clones; Y axis: Hybridization signal intensity ratio (normalized) of a sample to a reference.

### Example 9

Samples of genomic DNA template from human samples having known chromosome deletions are amplified and simultaneously labeled as described above. Parallel genomic hybridizations are then performed using the resulting labeled amplified DNA to analyze the test material compared to standard reference DNA. Statistical analysis is performed and indicates that the amplified genomic DNA is characterized by high fidelity of gene copy number profile with less than 10% gene copy number error and shows the expected chromosome deletions, evidenced by decreased hybridization signal intensity ratios.

In a particular example, a human syndrome sample with known deletion of a chromosomal region is used. The human DNA sample (CDC2 DNA) was purchased from Coriell Institute. Labeling, amplification, and hybridization (PGH) were performed as described herein, and reduced ratios across all five BAC clones derived from the CDC specific chromosomal region were observed, (0.61 indicative of one copy loss), while DNA BAC clones derived from all other regions have ratio of 1 (indicative of equal number of chromosome), except sex chromosome. FIG. 1B is a graph showing results of a parallel genomic hybridization (PGH) assay using amplified genomic DNA prepared as described herein, to demonstrate the >99% confidence of detecting single-copy loss of a chromosomal region (s). FIG. 1B: X-axis: BAC DNA probes (chromosomal regions carried by bacterial artificial clones; Y axis: Hybridization signal intensity ratio (normalized) of a sample to a reference.

### Example 10

Samples of genomic DNA template from human amniotic samples are amplified and simultaneously labeled as described above. Parallel genomic hybridizations are then performed using the resulting labeled amplified DNA to analyze the test amniotic samples compared to standard reference DNA. Statistical analysis is performed and indicates that the amplified genomic DNA is characterized by high fidelity of gene copy number profile with less than 10% acne copy number error. These results are in accordance with the diagnosis achieved based upon traditional karyotyping techniques.

Any patents or publications mentioned in this specification are incorporated herein by reference to the same extent as if each individual publication is specifically and individually indicated to be incorporated by reference.

The compositions and methods described herein are presently representative of preferred embodiments, exemplary, and not intended as limitations on the scope of the invention. Changes therein and other uses will occur to those skilled in the art. Such changes and other uses can be made without departing from the scope of the invention as set forth in the claims.

1. A method of amplifying genomic DNA, comprising:
contacting template genomic DNA, a plurality of random primers, and a DNA polymerase, wherein the ratio of template genomic DNA to random primers is in the range of about 1:10-1:35,000,000 (w/w), inclusive, to produce a reaction mixture; and
incubating the reaction mixture under isothermal conditions suitable for DNA synthesis, thereby producing amplified genomic DNA characterized by less than 10% gene copy number error.

2. The method of claim 1, wherein the template genomic DNA is in denatured condition.

3. The method of claim 1, wherein the template genomic DNA is not in denatured condition.

4. The method of claim 1, wherein the reaction mixture comprises detectably labeled nucleotides.

5. The method of claim 1, wherein the reaction mixture comprises detectably labeled random primers.

6. The method of claim 1, wherein the template genomic DNA is derived from a source selected from a human, a non-human mammal, a vertebrate, an invertebrate, a microorganism, a plant, cultured cells and laboratory manipulated cells.

7. The method of claim 1, wherein the template genomic DNA is amplified at least 3-fold.

8. The method of claim 1, wherein the template genomic DNA is amplified at least 2,500-fold.

9. The method of claim 1, wherein the template genomic DNA template is present in the reaction mixture in an amount in the range of about 3 picograms-1.5 micrograms, inclusive.

10. The method of claim 1, wherein the template genomic DNA template is present in the reaction mixture is representative of a single genome.

11. The method of claim 1, wherein the template genomic DNA template is present in the reaction mixture is representative of one or more chromosomes.

12. A method of assaying genomic DNA, comprising:
contacting template genomic DNA, random primers, and a DNA polymerase, wherein the ratio of template genomic DNA to random primers is in the range of about 1:10-1:35,000,000 (w/w), inclusive, to produce a reaction mixture;
incubating the reaction mixture under isothermal conditions suitable for DNA synthesis, thereby producing amplified genomic DNA characterized by less than 10% gene copy number error; and
comparing at least a portion of the amplified genomic DNA to a standard, thereby assaying genomic DNA.

13. The method of claim 12 wherein the amplified genomic DNA is compared to reference DNA to determine a number of copies of one or more regions in the amplified genomic DNA.

14. The method of claim 12, wherein the comparison is performed using a genomic hybridization method.

15. A method of producing detectably labeled amplified genomic DNA, comprising:
contacting genomic DNA template, random primers, dNTPs wherein at least one type of dNTP is detectably labeled, and a DNA polymerase, wherein the ratio of genomic DNA to random primers is in the range of about 1:10-1:35,000,000 (w/w), inclusive, to produce a reaction mixture; and
incubating the reaction mixture under substantially isothermal conditions suitable for DNA synthesis, thereby producing detectably labeled amplified genomic DNA characterized by less than 10 percent gene copy number error,

16. The method of claim 15, wherein the template genomic DNA is in denatured condition.

17. The method of claim 15, wherein the template genomic DNA is not in denatured condition.

18. The method of claim 15, wherein the reaction mixture comprises a detectably labeled nucleotide analog.

19. The method of claim 15, wherein the reaction mixture comprises detectably labeled random primers.

20. The method of claim 15, wherein the template genomic DNA is derived from a source selected from a human, a non-human mammal, a vertebrate, an invertebrate, a microorganism, a plant, cultured cells and laboratory manipulated cells.

21. The method of claim 15, wherein the template genomic DNA is amplified at least 3-fold.

22. The method of claim 15, wherein the template genomic DNA is amplified at least 2,500-fold.

23. The method of claim 15, wherein the template genomic DNA template is present in the reaction mixture in an amount in the range of about 3 picograms-1.5 micrograms, inclusive.

24. The method of claim 15, wherein the template genomic DNA template is present in the reaction mixture is representative of a single genome.

25. The method of claim 15, wherein the template genomic DNA template is present in the reaction mixture is representative of one or more chromosomes.

26. A method of amplifying genomic DNA, comprising:
contacting template genomic DNA, a plurality of random primers, and a DNA polymerase, wherein the ratio of template genomic DNA to random primers is in the range of about 1:20,000-1:35,000,000 (w/w), inclusive, to produce a reaction mixture; and
incubating the reaction mixture under isothermal conditions suitable for DNA synthesis, thereby producing amplified genomic DNA characterized by less than 10% gene copy number error.

27. The method of claim 26, wherein the reaction mixture comprises detectably labeled nucleotides.

28. The method of claim 26, wherein the reaction mixture comprises detectably labeled random primers.
